(19) United States
(12) Patent Application Publication
(10) Pub. No.: US 2008/0145323 A1
(43) Pub. Date: Jun. 19, 2008

(54) FORMULATION AND METHOD FOR A SUNBLOCK FOR ANIMALS

(75) Inventors: Thomas W. Dann, Oldsmar, FL (US); Michael C. Fleck, Bradenton, FL (US)

Correspondence Address: DLA PIPER US LLP, 4365 EXECUTIVE DRIVE, SUITE 1100, SAN DIEGO, CA, 92121-2133, US

(73) Assignee: Epiderma Pet, LLC, Bradenton, FL (US)

(21) Appl. No.: 11/852,098

(22) Filed: Sep. 7, 2007

Related U.S. Application Data
(60) Provisional application No. 60/843,498, filed on Sep. 7, 2006.

(51) Int. Cl. A61K 8/30; A61Q 17/04

(52) U.S. Cl. 424/59

(57) ABSTRACT

A composition for treating the skin of a mammal in need of protection from sun exposure is provided, the composition including at least one UV absorber, at least one skin conditioner, at least one film former, at least one antioxidant, and at least one fragrance. A method for preparing such a composition as well as a method for using the composition for protecting the skin of a mammal, such as a human or a domestic animal, e.g., a dog, a cat, or a horse, from sun exposure are also provided.

### CROSS-REFERENCE TO RELATED APPLICATIONS

This application claims priority under 35 U.S.C. § 119(e) to U.S. Patent Application Ser. No. 60/843,498 filed Sep. 7, 2006, which is herein incorporated by reference in its entirety.

### FIELD OF THE INVENTION

The present invention relates generally to mammal skin care products and more specifically to methods of using such products for topical treatment of mammals, including humans and pets the skin of which is in need of protection from sun.

### BACKGROUND

Many mammals, including humans and domestic animals, such as dogs, cats, and horses frequently suffer from exposure to sun, particularly when the exposure is prolonged. It is very desirable to have a product that can provide protection from sun radiation. Currently, such a product has not been provided. The present application provides the formulations that achieve these goals and describes the methods of using such compositions.

### SUMMARY

The present invention provides a composition useful for treating an animal having a skin in need of protection from sun radiation, the composition comprising at least one UV absorber, at least one skin conditioner, at least one film former, at least one antioxidant, (e) at least one fragrance, and at least one solvent.

The present invention further provides a method for preparing such a composition as well as a method for using the composition for protecting the skin of a domestic animal such as a dog, a cat, or a horse, from sun exposure.

### BRIEF DESCRIPTION OF DRAWINGS

FIGS. 1-3 demonstrate schematically a method of treating a domestic animal according to an embodiment of the present invention.

### DETAILED DESCRIPTION

According to an embodiment of the present invention, an aqueous composition is provided for treating mammals, including humans and animals, to achieve enhanced protection against sun radiation. The composition can include at least one UV absorber, at least one skin conditioner, at least one film former, at least one antioxidant, and at least one fragrance. The overall composition can contain a total of between about 10 and 25 mass % of all the above-mentioned components combined and the balance of at least one solvent, such as water or SD alcohol 40, or combination thereof. Optionally, the composition can also be formulated using any commonly used buffer system, if maintaining a certain level of pH is necessary.

The total concentration of the UV absorber(s) in the composition can be between about 20 and 30 mass % of the total composition, such as between about 24 and 28 mass %, for example, about 26 mass %. Examples of the UV absorber(s) that can be used include, but are not limited to, ethylhexyl methoxycinnamate, homosalate, benzophenone-3, and octyl salicylate, also known as ethylhexyl salicylate.

The total concentration of the skin conditioner(s) in the composition can be between about 1 and 5 mass % of the total composition. Examples of the skin conditioner(s) that can be used include, but are not limited to, C₁₂-C₁₅ alkyl benzoates.

The total concentration of the film former(s) in the composition can be between about 1 and 5 mass % of the total composition. Examples of the film former(s) that can be used include, but are not limited to, acrylates and octylacrylamide copolymers.

The total concentration of the antioxidant(s) in the composition can be between about 0.1 and 0.9 mass % of the total composition. An example of the anti-oxidant that can be used includes, but is not limited to, tocopheryl acetate. The total concentration of the fragrance(s) in the composition can be between about 0.1 and 0.9 mass % of the total composition.

Any composition satisfying the above-described requirements can be prepared using common formulating techniques known to those having ordinary skill in the art. For example, the above-described components can be mixed with one another, followed by adding water, to form an aqueous composition, e.g., by employing rapid stirring. Alternatively, each component, in a separate container, can be preliminary dissolved in water, or otherwise mixed with water resulting in a plurality of water-based systems, each contained in a separate container. The contents of all the containers can then be combined, e.g., by stirring or shaking, to form the final composition.

If desired, those having ordinary skill in the art can design other methods of mixing the components forming the composition. Regardless of the method of mixing that is selected, those having ordinary skill in the art will provide such quantities of each component so that the concentration of each of the components in the composition satisfies the above-described limits.

A method for the treatment of a mammal, such as a human or an animal, is further provided. A composition can be prepared according to a procedure described above. The composition can be then applied topically onto the skin of a mammal, such as a human or an animal, that needs the protection from sun exposure. Various methods can be used for applying the composition onto the skin of an animal. For example, the composition can be sprayed using a conventional hand-operated pump. Alternatively, the composition can be formulated to form an aerosol using commonly known methods for aerosol preparation. The composition can be also formulated as a solution or a shampoo. Those having ordinary skill in the art can devise other methods for applying the composition.

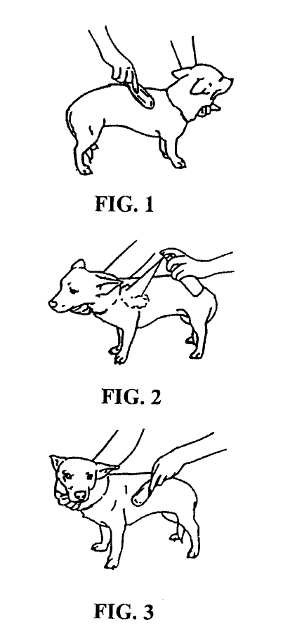

Referring now to FIGS. 1-3, one exemplary procedure that can be utilized for the treatment of a dog can be briefly described as follows. A suitable brush can be used for the procedure below, such as a two-sided brush that has metal bristles on one side and nylon-type bristles on the other. Using such or similar brush, the hair coat and skin of a dog can be energetically brushed using the metal side of the brush (FIG. 1). The duration of brushing can be at least 30 seconds.

Following the brushing procedure, the composition can be sprayed onto the entire body of the dog (FIG. 2). A hand-pump can be used for spraying. Alternatively, the composition can be applied using an aerosol generator, such as an aerosol can. At least 10 sprays can be used. The heaviest spraying can be applied over the back and sides of the dog. Fewer sprays can be applied for a smaller dog, and more sprays can be used if the dog is larger. The product should not be sprayed into the animal's eyes. Instead, the product can be applied to the brush first, followed by gentle brushing over the head and face.

When the product has been sprayed on, the product can then be brushed into the hair coat and skin of the dog using the metal side of the brush (FIG. 3). The duration of brushing can be at least 30 seconds. Then, the brush head can be reversed and the hair coat can be gently smoothed over. The soft side of the brush can be used for brushing the head and underside of the dog. If the dog's skin is red and sensitive, the softer side of the brush can be used. The entire procedure can be repeated daily.

A variety of mammals can be treated according to the methods of the present invention. In addition to dogs, as disclosed in the above-described exemplary procedure, some non-limiting examples of other animals that can be similarly treated can also include other domestic pets, such as cats, as well as horses, cows, sheep, goats, guinea pigs, rats or other bovine, ovine, equine, canine, feline, or rodent species. Humans may be also treated using the compositions and methods of the present invention. Those skilled in the art can adjust the treatment procedure taking in account the nature of the mammal to be treated.

The following example is provided to further illustrate the advantages and features of the present invention, but are not intended to limit the scope of the invention.

### EXAMPLE 1

### Preparation and Use of a Sunblock Composition

Composition # 1 was prepared by mixing, by energetic stirring for about three hours, the following components in the quantities shown below:

(a) about 7.5 mass % of UV absorber ethylhexyl methoxycinnamate;

(b) about 7.5 mass % of UV absorber homosalate;

(c) about 6.0 mass % of UV absorber benzophenone-3;

(d) about 5.0 mass % of UV absorber octyl salicylate;

(e) about 2.5 mass % of skin conditioner C₁₂ alkyl benzoate

(f) about 2.5 mass % of film former octylacrylamide copolymer;

(g) about 0.5 mass % of antioxidant tocopheryl acetate;

(h) about 10.0 mass % of water; and

(i) the balance, SD alcohol 40.

The resulting composition # 1 can be applied on a domestic animal in need of protection from sun radiation, such as a dog or a cat, using the procedure described above and illustrated by FIGS. 1-3.

Although the invention has been described with reference to the above example, it will be understood that modifications and variations are encompassed within the spirit and scope of the invention. Accordingly, the invention is limited only by the following claims.

What is claimed is:

1. A method for treating a mammal having a skin in need of protection from sun radiation, the method comprising topically administering to the mammal in need of said protection an effective amount of a composition comprising:
(a) at least one UV absorber;
(b) at least one skin conditioner;
(c) at least one film former;
(d) at least one antioxidant;
(e) at least one fragrance; and
(f) at least one solvent,
thereby treating the animal.

2. The method of claim 1, wherein the topical administering comprises spraying the composition on the skin of the mammal.

3. The method of claim 1, wherein the UV absorber is selected from the group consisting of ethylhexyl methoxycinnamate, homosalate, benzophenone-3, and octyl salicylate.

4. The method of claim 1, wherein the skin conditioner is selected from the group consisting of C₁₂-C₁₅ alkyl benzoates.

5. The method of claim 1, wherein the film former is selected from the group consisting of acrylates and octylacrylamide copolymers.

6. The method of claim 1, wherein the antioxidant is tocopheryl acetate

7. The method of claim 1, wherein the solvent is selected from the group consisting of SD alcohol 40 and water.

8. The method of claim 1, wherein the mammal is selected from a group consisting of a dog, a cat, and a horse.

9. The method of claim 1, wherein the mammal is a human.

10. The method of claim 1, wherein the topical administration comprises applying the composition by spraying using a pump or by applying an aerosol by using an aerosol generator, and brushing the composition onto the skin of the mammal.

11. A composition for treating a mammal having a skin in need of protection from sun radiation, comprising:
(a) at least one UV absorber;
(b) at least one skin conditioner;
(c) at least one film former;
(d) at least one antioxidant;
(e) at least one fragrance; and
(f) at least one solvent.

12. The composition of claim 11, wherein the UV absorber is selected from the group consisting of ethylhexyl methoxycinnamate, homosalate, benzophenone-3, and octyl salicylate.

13. The composition of claim 11, wherein the skin conditioner is selected from the group consisting of C₁₂-C₁₅ alkyl benzoates.

14. The composition of claim 11, wherein the film former is selected from the group consisting of acrylates and octylacrylamide copolymers.

15. The composition of claim 11, wherein the antioxidant is tocopheryl acetate

16. The composition of claim 11, wherein the solvent is selected from the group consisting of SD alcohol 40 and water.

17. A kit comprising packaging material and the composition according to claim 10 contained within the packaging material, wherein the packaging material comprises a label which indicates that the composition can be used for topical administration to a mammal having a skin in need of protection from sun radiation.

18. A method for manufacturing the composition according to claim 11, comprising mixing the components (a) through (f), wherein the composition comprises between about 20 and 30 mass % of the UV absorber.

19. The method of claim 18, wherein the composition further comprises between about 1 and 5 mass % of the skin conditioner.

20. The method of claim 19, wherein the composition further comprises between about 1.0 and 5.0 mass % of the film former.

21. The method of claim 20, wherein the composition further comprises between about 0.1 and 0.9 mass % of the antioxidant.

22. The method of claim 21, wherein the composition further comprises between about 0.1 and 0.9 mass % of the fragrance.
